(19) United States
(12) Patent Application Publication
(10) Pub. No.: US 2003/0205454 A1
(43) Pub. Date: Nov. 6, 2003

(54) Fluid mixing and irradiation device and method especially for biological fluids

(75) Inventors: Dennis Hlavinka, Arvada, CO (US); Michael A. Martinez, Golden, CO (US)

Correspondence Address: GAMBRO, INC, PATENT DEPARTMENT, 10810 W COLLINS AVE, LAKEWOOD, CO, 80215, US

(73) Assignee: Gambro, Inc., Lakewood, CO (US)

(21) Appl. No.: 10/427,260

(22) Filed: Apr. 30, 2003

Related U.S. Application Data
(60) Provisional application No. 60/377,697, filed on May 3, 2002.

(51) Int. Cl. B01J 19/08; C07C 1/00

(52) U.S. Cl. 204/157.15; 422/186.3

(57) ABSTRACT

The present invention relates to a method and/or apparatus suitable for use in reduction of any pathogens in a fluid such as a biological fluid, or a fraction or component thereof, which may contain pathogens. The device may include a vessel having an inlet and an outlet and a passage which extends therebetween. The passage may have a wall which is substantially transparent to a pathogen reduction radiation. The passage contains a static mixer system which is formed and arranged for thoroughly mixing the fluid in use of the device so as to bring substantially the whole of the fluid into an irradiation zone extending along and in substantially direct proximity to the passage walls during passage between the inlet and the outlet to be expose the fluid to a similar substantial level of irradiation. The static mixer may include light transmissive blades.

### RELATED APPLICATIONS

This application claims the benefit of priority from provisional patent application 60/377697 filed May 3, 2002.

### INTRODUCTION

The present invention relates to the treatment of fluids, especially biological or body fluids, such as human blood and fractions or components thereof to inactivate or reduce selected components, e.g. pathogens which may include microorganisms, viruses, bacteria and/or the like, and in particular relates to a fluid mixing and irradiation device or method suitable for use in such a pathogen reduction procedure.

### BACKGROUND

Large amounts of body fluids such as blood and plasma and various fractions or components thereof are used in the treatment of patients suffering from a variety of conditions. However, contamination of such fluids with various pathogens such as viruses and other microorganisms can give rise to serious new conditions in the patients receiving transfusion of these fluids and may even result in their death.

It has been found that fluids containing certain substances or agents are susceptible to be activated to reduce viruses, bacteria, and other microorganisms or pathogens. Some of these substances and agents are activatable by light radiation or irradiation for the reduction of pathogens. This light or photo-activation can be hindered somewhat by the opacity of the fluid into which the light is radiated. Thus, mixing of the fluid can be performed during irradiation to enhance radiation exposure. Frequently such mixing is done in a batch-wise procedure using a shaker table. Also, a flow-through system for pathogen reduction can be used. A pathogen reduction procedure and system is shown in U.S. Pat. No. 6,277,337.

Visible and/or ultra-violet (UV) irradiation can thus be used to activate certain substances or agents which thereby, when activated, work to reduce pathogens such as bacteria and viruses. However, this has been less practical with blood products because of the very low transmissibility of light into blood and hence the difficulty of ensuring a complete irradiation and inactivation or reduction. This problem is particularly pronounced with respect to a blood product or component having red blood cells.

In the past static mixers or flow splitters have been used for such industrial processes as epoxy mixing. Also, static mixers or flow splitters have been used for fluid flow mixing with an irradiation area. It is against this background that the instant invention was conceived.

### BRIEF SUMMARY OF THE INVENTION

In one aspect, the present invention provides a device suitable for use in the sterilization of, or reduction of any pathogens in a fluid, for example, a biological fluid or a component thereof potentially containing pathogens perhaps including lymphocytes and/or microorganisms. Such a device may include a vessel having an inlet and an outlet and a passage extending substantially directly therebetween to form a flow-through system, the passage having a wall which is substantially transparent to pathogen reduction radiation. A static mixer device may be formed and arranged in the passage for thoroughly mixing a fluid in use of the device, so as to bring substantially the whole of the fluid into an irradiation relationship with the wall extending along and between the inlet and the outlet. The static mixer may also include light transmissive blades or protrusions which may provide light penetration into the fluid flow path. Thus, in use of the device, substantially the whole of a body of the fluid passed through the vessel may be exposed to a similar substantial level of irradiation.

Hence, with a device of the present invention, a particularly uniform treatment of the fluid with respect to irradiation thereof may be achieved thereby avoiding under-exposure to pathogen reduction radiation whether as a result of screening by an excessive depth of relatively opaque fluid components or otherwise. Substantial mixing of the fluid to be treated with a pathogen reduction agent such as photosensitizers, if used to provide the pathogen reduction reaction, may also be provided. Either or both of these will then maximize reduction of any pathogens in the fluid.

A cylindrical form of vessel may be used, where in one embodiment, there may be an outer wall substantially transparent to pathogen reduction radiation and, in another embodiment, an inner wall being the substantially transparent wall or both the inner and outer walls may be transparent. Any or all of these may then be used with light transmissive static mixing elements or blades in a light communication relationship therewith.

In another aspect of the invention the device may include at least one pathogen reduction radiation source mounted in more or less closely spaced proximity to the transparent wall. Note that transparency for the transparent wall and/or the static mixing elements is intended to indicate substantial transmission of radiation at a desired photo-activation wavelength, which may or may not be accompanied by significant transparency at other wavelengths, e.g., visible or UV light. The mounting of the radiation source may generally be arranged to maximize the radiation intensity in the irradiation zone. Indeed high transmissivity may also be made or further maximized through the static mixer blades as well.

One or various pathogen reduction radiation wavelengths may be used and this may depend upon a particular pathogen reduction agent or photosensitizer, if used, or may depend on the blood component or bodily fluid to be irradiated. For example, riboflavin may be used and this may suggest using radiation having a wavelength range from about 300 nm to about 500 nm, for example, or perhaps more appropriately at about 447 nm for red blood cells. Another example of a photo-activatable agent that may be used is a psoralen, e.g., 8-methoxy psoralen, which upon exposure to UV radiation of from about 320 to about 400 nm wavelength may become capable of forming photoadducts with DNA in lymphocytes to thereby reduce or inactivate these. Various light source types may be used whether of the fluorescent type, incandescent lamps or LED's, inter alia.

Where light radiation is used to effect inactivation or reduction of pathogens, then the vessel side wall (inner or outer or both) and/or the static mixer blades may be made of various light transmissive materials including for example silica and other glasses; silicones; quartz, cellulose products and plastics materials such as polytetrafluoroethylene (PTFE), fluorinated ethylenepropylene (FEP), and/or low density polyethylene (LDPE) or polyvinyl chloride (PVC).

Other inactivating or reducing radiation wavelengths that may be used include microwave radiation used in conjunction with a glass or ceramic vessel wall or blades; infrared radiation used in conjunction with a quartz vessel wall or blades. The duration of irradiation required will depend on various factors such as the intensity, disposition and number of sources used, the transmission characteristics of the vessel side wall or blade material, the vessel configuration and hence the mixing efficiency therein and the surface area of the thin layer of fluid adjacent the vessel side wall and/or mixing blades, the length of the passage in the vessel and the flow rate of the fluid being treated, and hence the residence time of the fluid in the irradiation zone, as well as the nature of the fluid itself. In general, the residence time in the vessel, the material and thickness of the vessel side wall and/or blades and the radiation sources may be chosen and arranged to provide an effective reducing or inactivating dosage of radiation within such a period.

The required irradiation time can be achieved in a number of different ways including one or more of the following: use of vessels with irradiation zones of different length, varying the flow rate of the fluid, using a plurality of devices in series or parallel, and recycling the fluid through the device(s) a number of times.

It will also be understood that the degree of mixing desired to achieve complete irradiation may depend on various factors such as the transmissibility or permeability of the fluid to the radiation and the total depth of fluid in the vessel from the wall or blades through which radiation is received. In general the lower the transmissibility/permeability and the greater the fluid depth, the greater will be the number of mixer blade elements and mixing stages desired for effective irradiation.

### BRIEF DESCRIPTION OF THE DRAWINGS

Further features and advantages of the invention will appear from the following detailed description given by way of examples and illustrated with reference to the accompanying drawings in which: FIG. 1 is a partly schematic, partly cross-sectional view of an irradiation apparatus according to the present invention;

FIG. 2 is a partly schematic, partly cross-sectional view of an alternative embodiment according to the present invention;

FIG. 3 is a transverse section of an alternative embodiment of the invention using LED's;

FIG. 4 is a schematic view of an alternative apparatus according to the present invention;

FIG. 5 is a cut-away, cross-sectional view of the apparatus of FIG. 4;

FIG. 6 is an isometric view of an apparatus such as that shown in FIG. 4; and

FIG. 7 is a schematic view showing flow through a further alternative apparatus according to the present invention.

### DETAILED DESCRIPTION

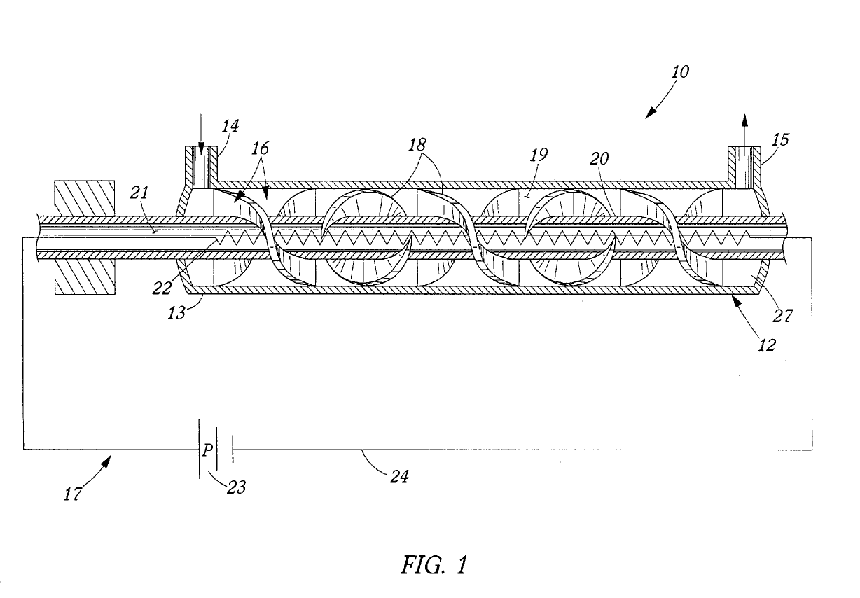

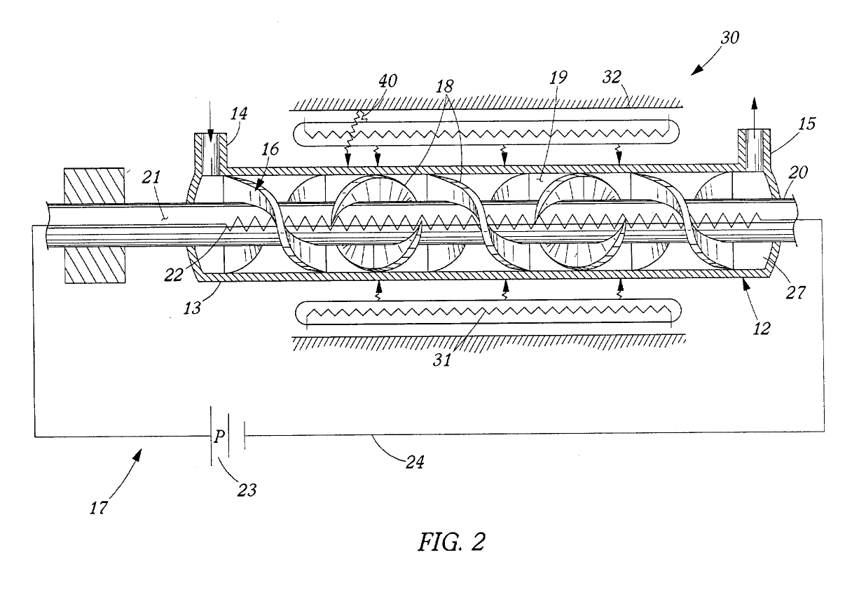

FIG. 1 shows an apparatus 10 comprising a vessel 12 in the form of a cylindrically walled tube 13. In one embodiment (see FIG. 2), tube walls 13 may be of a light transmissible material. Vessel 12 also has an inlet 14 and an outlet 15, with an axially extending static mixer device 16 disposed within the walls 13 thereof. In more detail the static mixer device 16 comprises an axially extending series of angularly offset helical “screw” or paddle or blade elements 18 defining pairs of flow paths which are divided equally and mixed at the junctions 19 between successive elements 18 thereby providing a degree of mixing which increases with the number of elements used. Blades 18 may also be referred to as flow splitting elements wherein they may split flow streams in a repeating fashion. Another effect of the blades 18 is to move the streams adjacent an outer wall 13 or walls (of a core 20), as will be described.

The “screw” or blade elements 18 may be, as shown in FIG. 1, mounted on a hollow core 20 which defines one embodiment of light transmission. In particular, a passage 21 in core 20 forms a container for light source(s) 22 (shown schematically). In more detail, the light system 17 may include an electrical light circuit 24 provided with power supply 23 for circulating electricity for the lights 22 mounted inside the hollow core 20.

The walls of core 20, and in one embodiment, also the screw elements 18, may be made of an inert physiologically acceptable light conductive material such as transparent plastic in order to facilitate efficient light transfer from the light circuit 17 to the fluid being treated 27 to thereby maximize the exposure of the fluid to light by “turning over” fluid closely adjacent the core 20 and screw elements 18, so as to thereby maximize the efficiency of the sterilization/pathogen reduction treatment of the fluid. Light may thus be transmitted from a bulb or bulbs 22 through the walls of core 20 and into the flow passages between core 20 and walls 13 as well as, in one embodiment, into and through blades 18. The walls of core 20 represent the inner walls of the fluid flow passage of vessel 12 with walls 13 representing the outer walls.

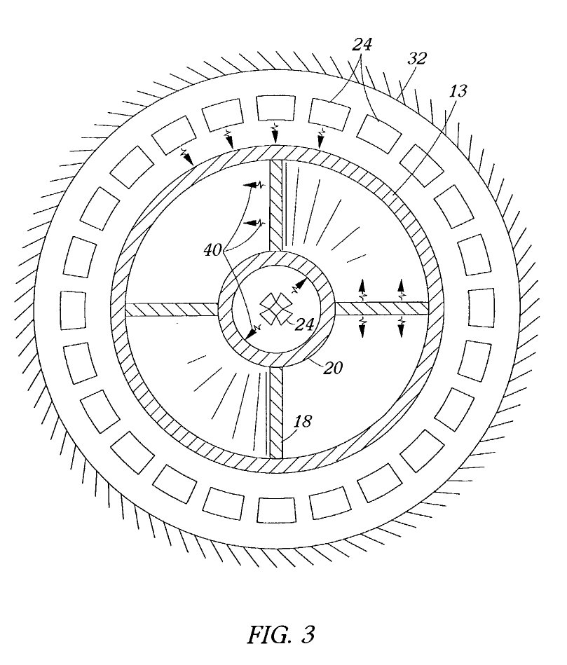

Irradiation (not separately shown in FIG. 1) may be effected by means of one or a plurality of light sources 22 (represented schematically in FIG. 1) inside the core 20. These light sources may be incandescent or fluorescent bulbs or tubes, or they may be a number of LED light sources (see FIG. 3).

In another embodiment, as shown in apparatus 30 of FIG. 2, additional or alternative exterior light sources 31 may be alternatively or additionally used. These may also be incandescent or fluorescent tubes or bulbs 31 (also shown schematically) and may extend parallel to and/or may be closely spaced from the vessel walls 13 and distributed there around. The light or irradiation sources could also be LED's or light emitting diodes arranged around and along the length of vessel walls 13 as shown in FIG. 3. Vessel walls 13 could then also be light transmissive, and in one embodiment, light transmissive screw blades 18 may be in light conduction communication herewith to convey light from sources 31 through walls 13 into the interior of mixer 16. Exterior reflectors 32 may also be provided to help concentrate the radiation 40 onto and through the vessel walls 13. The vessel walls 13 may be made of any of a number of light transmissive materials to maximize transmission of the radiation 40 into the fluid 27 being treated. Note, the inner core 20 with interior light source(s) 22 may continue to be used in addition as well. In this or any of the embodiments herein, flow through the system may be affected by movement of the system by a number of methods such as by vibration or gyration or nutation or gravity or pumping, including pumping in opposite directions, inter alia, to further increase mixing.

A cross-sectional view of another embodiment is shown in FIG. 3. In this FIG. 3 embodiment, another view of an apparatus is depicted in which the parts mostly correspond to those shown in the embodiment of FIG. 2. However, the light source(s) in this apparatus may include an array of light emitting diodes, LED's 24 (either or both inner and/or outer) each providing a desirable wavelength of electromagnetic radiation. The LED's 24 may be arranged so that angularly distributed LED's are positioned around the vessel tube 13 and along its length, as well as or alternatively may so be disposed inside the hollow core 20 along its length. Light rays 40 are shown here also, from both inner and outer LED's 24 as well as emanating from light transmissive blades 18, penetrating deeper into the fluid flow.

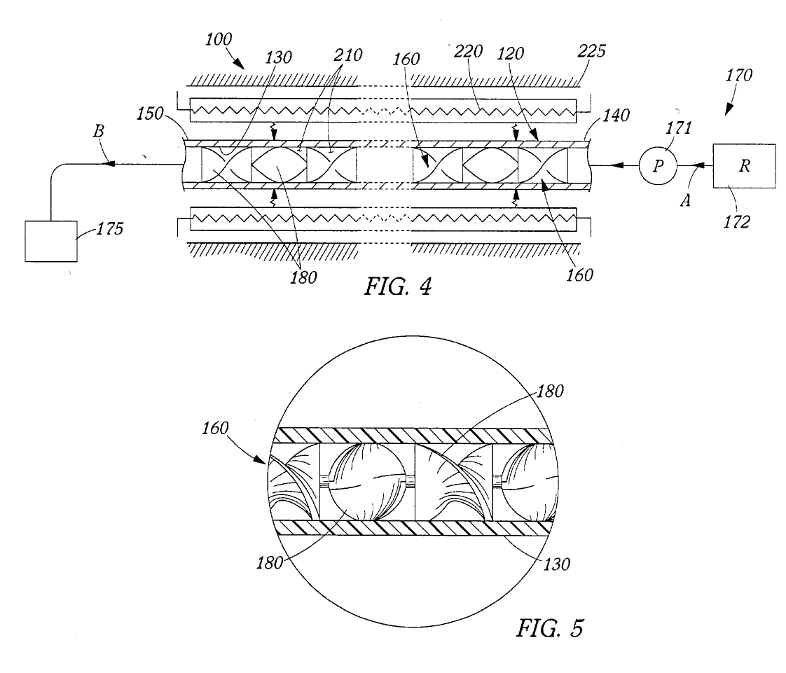

FIG. 4 shows an alternative apparatus 100 of the present invention comprising a tubular vessel 120 having a first end with an inlet 140 and a second end having an outlet 150. Arrow A shows the direction of flow of the liquid into the device and arrow B indicates the direction of the flow of the liquid exiting the device during use. A fluid flow supply 170 may be provided to pass fluid through the tubular vessel 120 in use of the apparatus. The fluid supply 170 may typically be a pump 171 which can pump the fluid through the device at a desired flow rate, for example, a peristaltic pump or a gear pump. In an alternative arrangement, the fluid may be supplied to the device 100 by arranging a reservoir 172 (reservoir discretely shown as a box) of the fluid to be held at a level substantially above the level of the inlet 140 and outlet 150 of the device 100. This arrangement may then allow the fluid to flow under the influence of gravity from the reservoir 172 through the tubular vessel 120 to the outlet 150 positioned below the level of the reservoir 172. Supply 170 may thus include one or the other or both pump 171 and/or reservoir 172. A receiver/container 175 is shown at the receiving end past outlet 150 of device 100. Reservoir 172 and receiver 175 may be conventional containers such as bags.

Although fluid is shown flowing through apparatus 100 in one direction it is also understood that the direction of fluid flow could be reversed to provide fluid flow in the opposite direction. Also, fluid flow could alternatively change direction periodically over time to provide further mixing and additional irradiation.

The tubular vessel 120 of the apparatus 100 may be in the form of a transparent tube wall 130. The tubular vessel may thus be substantially cylindrical. A static flow mixer 160 may be disposed in and extend along the length of the vessel 120 and may include a series of mixer elements 180 arranged longitudinally therein with pairs of alternatively handed screw elements or blades angularly offset from each other by some degrees, for example ninety degrees (90°). The mixer device 160 and blades 180 may be transparent and may have an outside diameter which meets the inner diameter of the tube walls 130, and may thus be push-fit inside the transparent tube vessel 120. A tight fit between the tube wall 130 and the blades 180 is desired such that fluid does not flow between the wall 130 and blade 180 and so that light can pass through the wall 130 and into the blade 180. Such a tight fit is desirable in all embodiments.

The mixer elements 180 in such devices may be formed and arranged such that in use the fluid may be thoroughly mixed so that different portions of the main body of the fluid are successively brought within a more or less shallow irradiation zone 210 adjacent the wall 130 of the vessel 120 to be light- irradiated. In this way substantially all of the fluid is exposed to a similar pathogen reduction level of light irradiation. With substantially light transmissive mixer elements 180, light may be transmitted deeper into the fluid flow and thereby provide greater exposure of the fluid to light.

Various angularly distributed light lamps 220 mounted inside a reflective housing 225 are positioned more or less closely adjacent around the vessel wall 130. In relation to control of the exposure of the fluid to visible or UV radiation, this is conveniently monitored in terms of the residence time of a fluid within any part of the transparent wall tubular vessel 120 between the opposed lamps 220, referred to herein as the irradiation area though it will be appreciated that the actual period of time during which any part of the fluid is actually irradiated—corresponding to residence time within the irradiation zone adjacent the walls of the vessel may be rather less than the residence time in the irradiation area, the difference depending on factors such as the outside diameter (OD) of the fluid and the diameter of the vessel as discussed hereinbefore.

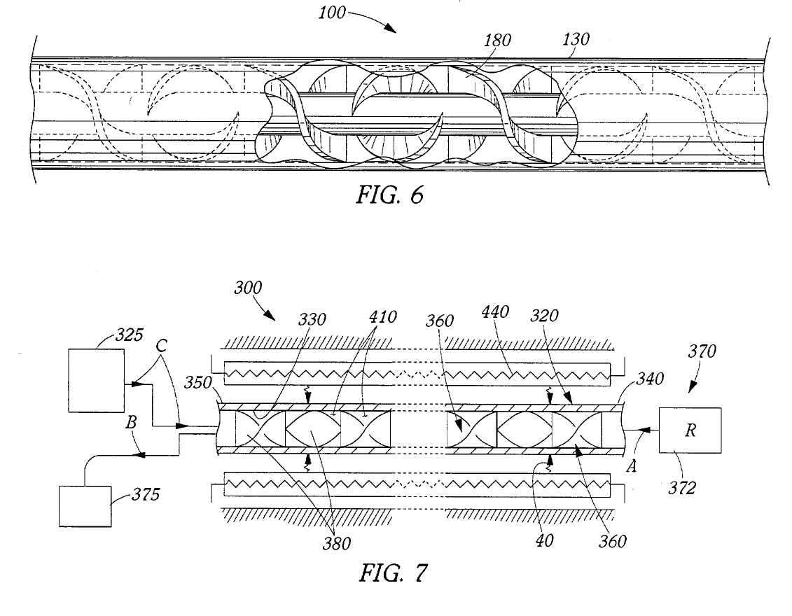

The amount of fluid in contact with or close proximity to the vessel wall 130 may usually be relatively small compared to the total volume of fluid present in the tubular vessel 120 at any given time. The fluid may be very thoroughly remixed as it passes from one mixer element 180 to the next. This may heighten the exposure of the components of the fluid to irradiation. A close-up example of what the blades 180 may look like in the device of FIG. 4 is shown in FIG. 5, and a further more isometric view with cut away portion is shown in FIG. 6 with like elements having like numbers with FIG. 4.

FIG. 7 shows an alternative embodiment which may also provide for mixing the fluid of interest with a gas (such as oxygen (O₂), nitric oxide (NO₂), or air, inter alia). The system 300 of FIG. 7 includes a flow vessel 320 with a vessel wall 330 having an inlet 340 and an outlet 350. A mixing device 360 is disposed inside the vessel 320 and may be adjacent the vessel wall 330. The mixing device 360 may have a plurality of blades 380 as shown and described in the embodiments of either FIGS. 1-3 and/or FIGS. 4 and 5. A further gas container 325 may be disposed as shown downstream of the flow-through vessel 320 (it may alternatively be disposed upstream thereof (not shown)). Also, the fluid flow may be orientated downward (either by gravity as shown or by pump) from a reservoir system 370, e.g., a reservoir 372 and as the fluid flow is downward, for example, the gas may be flowing or trickling upward, see flow arrow C, to provide enhanced mixing of the gas and fluid. This may be beneficial for certain uses such as where a pathogen reduction agent may be aided in operation by chemical combination of the gas therewith. For example, riboflavin as a pathogen inactivation or reduction agent may be further activated by combination with an oxygen product (e.g., oxygen (O₂), nitric oxide (NO₂), or air, inter alia). Thus fluid containing a photosensitizer may flow from one direction while the gas or oxygen flows into core or vessel 320 from another direction. Alternatively, both the gas and fluid can flow in the same direction. Horizontal or other 3-D space orientations may also be provided. Irradiated fluid may then be collected in a container 375 (though, it may alternatively be collected in the same chamber/container from which the gas was/is released, e.g., container 325).

In this embodiment, as was described for the previous embodiments above, the blades 380 may be of a light transmissive material to provide good penetration of light radiation into the fluid flow. Light radiation 40 may thus emit from source(s) 440 and irradiate fluid in an irradiation zone 410. Another alternative usable herewith may be to have a hollow core (not shown) such as that shown in FIGS. 1-3 with light source(s) disposed therein to irradiate the inner surface of fluid flow.

As noted relative to gas mixing, the static mixers of the present invention may also be used for mixing an agent, such as a pathogen reduction agent, with the fluid of interest (e.g., blood or a component thereof). Very thorough mixing of the agent with the fluid of interest may then provide enhanced exposure of all or substantially all of the fluid of interest with the agent. Thorough pathogen reduction may then result. Note, irradiation may be performed simultaneously, or before or after such agent mixing. Further, a pathogen reduction agent may originally be disposed in one or more discrete parts prior to use (for sterilization reasons, inter alia), and these may then be appropriately mixed using devices or systems of the present invention, before, simultaneously with or after mixture with the fluid of interest (e.g., blood or a component thereof).

The examples of the above-described systems, methods, and apparatuses are for illustrative purposes only. For example, although a cylindrical or annular vessel is described, it is understood that the outer vessel can be any shape, particularly if the static mixer is arranged in an inner passage. Because other variations will become apparent to those skilled in the art, the present invention is not intended to be limited to the particular embodiments described above. Any such variations and other modifications, adaptations or alterations are included within the scope and intent of the invention.

1. A device suitable for use in the irradiation of a fluid, which is a biological fluid or a fraction or component thereof that may contain pathogens, which device comprises:
an irradiation source for irradiating the fluid;
a vessel having
an inlet;
an outlet; and
a passage extending substantially directly through the vessel between the inlet and outlet therebetween;
said passage having a wall substantially transparent to pathogen reduction radiation from the irradiation source;
a static mixer device contained in the passage and formed and arranged for providing penetration of irradiation into the fluid and for thoroughly mixing the fluid in use of the device, so as to bring substantially the whole of the fluid into an irradiation zone extending along and in substantially direct proximity to said substantially transparent passage wall during fluid passage between said inlet and said outlet;
whereby in use of the device substantially the whole of a body of said fluid passed through said vessel may be exposed to a similar substantial level of irradiation.

2. A device as in claim 1 wherein the irradiation source is disposed in the interior of said passage.

3. A device as in claim 1 wherein said device further comprises
a conduit disposed inside the passage, and
said source of irradiation comprises interior light sources disposed within the conduit to emit light in the interior of said static mixer device.

4. A device as in claim 1 wherein said vessel has an annular form with an outer wall substantially transparent to irradiation.

5. A device as in claim 1 wherein the static mixer is substantially light transmissive.

6. A device as in claim 1 wherein said irradiation source comprises an irradiation source mounted in more or less closely spaced proximity to said substantially transparent passage wall.

7. A device as in claim 1 wherein said irradiation source emits an ultra violet radiation having a wavelength in the range of from about 200 to about 400 nm.

8. A device as in claim 1 wherein said irradiation source emits visible radiation having a wavelength in the range of from about 400 to about 500 nm.

9. A device as in claim 1 which is adapted to receive a pathogen reduction agent and mix said pathogen reduction agent with the biological fluid or component thereof.

10. A device as in claim 1 which is adapted to receive an alloxazine and mix said alloxazine with the biological fluid or component thereof.

11. A device as in claim 1 which is adapted to receive riboflavin and mix said riboflavin with the biological fluid or component thereof.

12. A device as in claim 1 which is adapted to receive psoralen and mix said psoralen with the biological fluid or component thereof.

13. A device as in claim 1 wherein said substantially transparent wall of said passage is made of a substantially light-transparent material selected from the group consisting of light-transparent glasses, silicone, quartz, cellulose products, and plastics materials.

14. A device as in claim 1 wherein the irradiation source is selected from the group consisting of microwave radiation used in conjunction with a glass passage wall; and infrared radiation used in conjunction with a quartz passage wall.

15. A device as in claim 1 further comprising reflectors spaced around the passage and formed and arranged to concentrate the radiation onto said passage wall.

16. A device as in claim 1 in which the vessel is adapted to receive a gas for mixing with the biological fluid or a fraction or component thereof.

17. A device as in claim 1 wherein the vessel further comprises a vessel outer wall substantially transparent to irradiation.

18. A device as in claim 17 wherein the irradiation source comprises
a first source of irradiation inside the passage,
a second source of irradiation outside the vessel and proximate to the vessel substantially transparent wall.

19. A device as in claim 1 wherein the irradiation source comprises a plurality of light emitting diodes.

20. A device as in claim 19 wherein the light emitting diodes are arranged inside the passage.

21. A device as in claim 19 wherein the vessel further comprises
an outer wall substantially transparent to irradiation and wherein the light emitting
diodes are arranged around and along the length of the outer wall.

22. A device as in claim 21 wherein the passage substantially light transmissive wall forms a conduit and wherein light emitting diodes are further arranged in the conduit.

23. A device as in claim 1 wherein the irradiation source is disposed exterior to the passage.

24. A device as in claim 1 wherein the static mixer device comprises blade elements.

25. A device as in claim 24 wherein the blade elements are transmissive to irradiation.

26. A device as in claim 24 wherein the blade elements are angularly offset helical blade elements.

27. A device as in claim 26 wherein the blade elements extend axially in the passage.

28. A fluid flow device for passing a fluid which is a biological fluid or a fraction or component thereof that may contain pathogens such that the fluid is adapted to be irradiated during passage through the fluid flow device comprising
an inlet for receiving the fluid;
an outlet for disposing of the fluid after passage through the fluid flow device;
a passage extending between the inlet and outlet for passing the fluid comprising
at least one light transmissive wall; and
a static mixer in the passage comprising
a plurality of angularly offset blade elements arranged along the passage for providing penetration of irradiation into the fluid and for thorough mixing of the fluid.

29. A fluid flow device as in claim 28 wherein the blade elements are formed of light transmissive material.

30. A fluid flow device as in claim 28 wherein the blade elements are helically arranged along the passage.

31. A method of irradiating a biological fluid or fraction thereof, that may contain pathogens for sterilizing or reducing any pathogens that may be contained in the biological fluid comprising the steps of:
passing the biological fluid through a passage so that the whole of a body of said fluid is exposed to a similar substantial level of pathogen reduction irradiation;
statically mixing the biological fluid in the passage during the passing step to provide the fluid to an irradiation zone; and
irradiating the fluid in the irradiation zone and into the biological fluid or fraction thereof to reduce any pathogens that may be contained therein.

32. A method as in claim 31 which includes a step, prior to passing said fluid through said passage, of incorporating into the fluid to be sterilized a photo-activatable agent, said agent being convertible from a non-activated form into a pathogen-reduction form by irradiation.

33. A method as in claim 31 which includes a step, prior to the passing step of incorporating into the fluid to be sterilized an alloxazine, said alloxazine being convertible from a non-activated form into a pathogen-reduction form by irradiation.

34. A method as in claim 31 which includes a step, prior to the passing step s of incorporating into the fluid to be sterilized riboflavin, said riboflavin being convertible from a non-activated form into a pathogen-reduction form by irradiation.

35. A method as in claim 31 which includes a step, prior to the passing step, of incorporating into the fluid to be sterilized a psoralen, said psoralen being convertible from a non-activated form into a pathogen-reduction form by irradiation.

36. A fluid flow device for passing a fluid comprising a biological fluid or a fraction or component thereof that may contain pathogens, and riboflavin such that the fluid is adapted to be irradiated during passage through the fluid flow device to activate the riboflavin to reduce pathogens in the biological fluid or a fraction or component thereof comprising
an inlet for receiving the fluid;
an outlet for disposing of the fluid after passage through the fluid flow device;
a passage extending between the inlet and outlet for passing the fluid comprising
at least one light transmissive wall; and
a static mixer in the passage comprising
a plurality of angularly offset blade elements arranged along the passage for providing thorough mixing of the fluid to bring substantially all of the fluid substantially proximate to the at least one light transmissive wall.
